(19) United States
(12) Patent Application Publication
(10) Pub. No.: US 2024/0175070 A1
(43) Pub. Date: May 30, 2024

(54) ANALYSIS APPARATUS AND ANALYSIS METHOD

(71) Applicant: CANON KABUSHIKI KAISHA, Tokyo (JP)

(72) Inventors: KEIGO MIZUSAWA, Kanagawa (JP); TAKESHI YAMAMOTO, Kanagawa (JP)

(21) Appl. No.: 18/499,454

(22) Filed: Nov. 1, 2023

(30) Foreign Application Priority Data
Nov. 9, 2022 (JP) 2022-179857
Sep. 1, 2023 (JP) 2023-142316

(51) Int. Cl. C12Q 1/527; B01L 3/00

(52) U.S. Cl. CPC C12Q 1/527; B01L 3/502746; B01L 2200/16; B01L 2300/0864

(57) ABSTRACT

It is an object to efficiently analyze a measurement object having a certain functional group. Provided is an analysis apparatus for analyzing a specimen that may contain one or more kinds of measurement objects each having a certain functional group, the analysis apparatus including: an addition unit to which the specimen is added; and a plurality of flow paths communicating downstream of the addition unit, and a plurality of detection units respectively communicating to the plurality of flow paths, wherein each of the plurality of detection units includes one kind of detection reagent that reacts with the certain functional group, and wherein the plurality of detection units include two or more kinds of detection reagents that each react with the certain functional group.

### BACKGROUND OF THE INVENTION

### Field of the Invention

The present invention relates to an analysis apparatus and analysis method for a measurement object having a certain functional group.

### Description of the Related Art

Antibodies recognize various antigens with high specificity. The high specificity of an antibody can be utilized to detect and quantify an antigen with high specificity. As an analysis method using an antibody, there are known numerous immunoassay methods including an ELISA method. In addition, the high specificity of an antibody has been utilized to establish a small and highly accurate analysis apparatus such as one for immunochromatography.

Meanwhile, a low-molecular-weight compound or the like may be detected with a reagent that specifically reacts with a functional group thereof. However, such reagents do not have high specificity like that of antibodies, and are often incapable of distinguishing between and recognizing compounds having the same functional group but having different structures. Accordingly, the low-molecular-weight compound or the like is often analyzed based on a difference in molecular weight or chemical properties.

For example, analyses of amino compounds, thiol compounds, carboxylic acids, carbonyl compounds, and the like are widely performed in medical tests, environmental tests, food tests, and the like, and high-performance liquid chromatography (HPLC) is widely used for those analyses. In analysis by HPLC, a difference in mobility in a column resulting from a difference between chemical properties of compounds is utilized to separate components, which are detected and quantified with a detector of absorbance, electrochemistry, mass spectrometry, fluorescence, chemiluminescence, or the like.

In HPLC, it is known that a compound that itself cannot be or is not sufficiently detected with the detector is enhanced in sensitivity to the detector by adding, via a functional group, a compound showing a high absorbance, a compound that emits fluorescence, or the like (Talanta 2003, 60, 1085-1095).

Analysis of a thiol compound, which is a compound having a thiol group, is utilized in a wide range of fields, for example, medical tests, such as determination of in vivo oxidative stress and diagnosis of thrombosis, environmental tests, and food tests. The analysis of the thiol compound is performed for the purpose of, for example, a soil or water test in a factory, an industrial waste treatment plant, or a sewage treatment plant and a surrounding area thereof, a test of the freshness or manufacturing process of food in the food industry, analysis of kinetics in a living body or living cells as a physiologically active substance, or a bad breath test. An example of an analysis method for the thiol compound is the above-mentioned high-performance liquid chromatography (HPLC).

However, the thiol compound is liable to be oxidized, and is converted into a symmetrical disulfide compound or an unsymmetrical disulfide compound through formation of a disulfide bond by a reaction between thiol groups. Such disulfide compound has lost the thiol groups, and hence is difficult to derivatize. To solve this problem, there is known a method involving performing a reduction reaction with a reducing agent before derivatization to convert the disulfide compound into the thiol compound, and then performing a derivatization reaction, followed by HPLC analysis (Japanese Patent Application Laid-Open No. 2008-185364). Alternatively, it is known that, after component separation with an HPLC column has been performed, a separated component is passed through a reducing column, and a fluorescence reagent solution is allowed to flow in the passed fluid to perform derivatization, followed by detection based on fluorescence (Japanese Patent No. 5119554). However, in such technique, a difference in mobility in the column may be hardly obtained depending on an impurity in a sample or the composition or concentrations of thiol compounds therein, leading to insufficient separation. Such measurement involves an even larger number of operation steps to complicate the analysis.

It is an object to efficiently analyze a measurement object having a certain functional group.

### SUMMARY OF THE INVENTION

A detection reagent that reacts with a certain functional group has been difficult to use for analysis of each of measurement objects having the same functional group but having different structures. Meanwhile, detection reagents that react with the same functional group but have slightly different reactivities with each of the measurement objects are easy to obtain. The inventors have focused their attention on the use of a plurality of such detection reagents, and have reached the present invention. That is, the present invention provides an analysis apparatus for analyzing a specimen that may contain one or more kinds of measurement objects each having a certain functional group, the analysis apparatus including: an addition unit to which the specimen is added; and a plurality of flow paths communicating downstream of the addition unit, and a plurality of detection units respectively communicating to the plurality of flow paths, wherein each of the plurality of detection units includes at least one kind of detection reagent that reacts with the certain functional group, and wherein the plurality of detection units include two or more kinds of detection reagents that each react with the certain functional group.

Further features of the present invention will become apparent from the following description of exemplary embodiments with reference to the attached drawings.

### BRIEF DESCRIPTION OF THE DRAWINGS

FIG. 1 is an illustration of an example of an analysis apparatus.

FIG. 2A, FIG. 2B, FIG. 2C, and FIG. 2D show examples of analysis results.

FIG. 3 is an explanatory diagram of an information processing device.

FIG. 4 is a flowchart for illustrating the steps of an analysis method.

FIG. 5 is a flowchart for illustrating the steps of an analysis method.

FIG. 6 is a flowchart for illustrating the steps of an analysis method.

FIG. 7E, FIG. 7F, FIG. 7G, and FIG. 7H show examples of analysis results.

### DESCRIPTION OF THE EMBODIMENTS

According to a first embodiment of the present invention, there is provided an analysis apparatus for analyzing a specimen that may contain one or more kinds of measurement objects, the measurement objects each being a substance that satisfies at least any one of the following conditions α) and β: α) having a certain functional group; and β) being capable of reacting with an enzyme to produce a compound having the certain functional group, the analysis apparatus including: an addition unit (an injection unit) to which the specimen is added; and a plurality of flow paths communicating downstream of the addition unit, and a plurality of detection units respectively communicating to the plurality of flow paths, wherein each of the plurality of detection units includes one kind of detection reagent that reacts with the certain functional group, wherein the detection reagent contains a compound having specific reactivity with the certain functional group, and wherein the plurality of detection units include two or more kinds of detection reagents different from each other in the reactivity.

### Analysis Apparatus

According to the analysis apparatus of this embodiment, the specimen added to the addition unit reaches the plurality of detection units via the plurality of flow paths communicating downstream of the addition unit through utilization of the driving force of a pump, capillarity, or the like. Meanwhile, each of the plurality of detection units includes a different kind of detection reagent that reacts with the functional group. The measurement object in the specimen that has reached each detection unit reacts with the detection reagent included in the detection unit. Then, the measurement object in the specimen can be analyzed based on signal information emitted from the plurality of detection units. It is preferred that the analysis apparatus according to this embodiment be a fluidic device itself or include a fluidic device. The “fluidic device” is a product obtained by forming a flow path pattern on a base material. The fluidic device is described later. The “analysis” refers to detection, identification, qualitative analysis, or quantitative analysis of a substance, or any other form of obtainment of information about a substance of interest.

The analysis apparatus according to this embodiment is used alone or in combination with another apparatus or the like as, for example, an automatic blood analyzer for clinical use, a simple test instrument for medical use, a test apparatus for rapid diagnosis, a biochemical test apparatus, a lateral-flow test chip, a flow-through test chip, a dipstick, a microfluidic chip, a microchemical chip, a biochip, or a fluidic device.

### <Measurement Object>

The measurement object is a substance that satisfies at least any one of the following conditions α) and β):
α) having a certain functional group; and
β) being capable of reacting with an enzyme to produce a compound having the certain functional group.

The certain functional group is not particularly limited, and examples thereof include a thiol group, an amino group, a carboxy group, a carbonyl group, and a hydroxy group. The measurement object having the certain functional group is a compound having any of those functional groups, and examples thereof include an amino compound, a thiol compound, a carboxylic acid, a carbonyl compound, and an alcohol compound.

In addition, the measurement object may be, for example, an amide compound, a peptide compound, a thioether compound, an ether compound, a glycoside compound, an ester compound, or a phosphoric acid compound, which can be converted into the compound having the certain functional group through, for example, a hydrolysis reaction using an enzyme. Examples of the enzyme to be used in the production of the compound having the certain functional group include enzymes, such as a peptidase, an esterase, a glycosidase, a lyase, and a phosphatase. The measurement object can be detected by mixing the specimen with the enzyme to convert the measurement object into a substance having the certain functional group, and allowing the substance after the conversion to react with the detection reagent. That is, a substance that, though not being a compound having the certain functional group, produces a compound having the certain functional group through an enzymatic reaction may be used as the measurement object.

Particularly in an embodiment including a reduction unit, the thiol compound is a suitable measurement object. Examples of the compound having a thiol group include proteins and hydrogen sulfide ions, and compounds, such as cysteine, homocysteine, glutathione, methanethiol, ethanethiol, cysteamine, mercaptopyruvic acid, and hydrogen sulfide. Of those, cysteine, homocysteine, glutathione, methanethiol, and the like are suitable measurement objects as biomarkers related to a disease of a living body, a physical condition thereof, the degree of stress applied to a living body, and the like, compounds in an environment, various compounds derived from a living body, and various compounds contained in a food and drink. Methionine, which can be converted into methanethiol using methionine γ-lyase, is also a suitable measurement object.

The specimen that may contain the measurement object is not particularly limited, and encompasses, for example, a biological sample, such as urine, blood, sweat, tears, saliva, or mucus, an environmental sample, a food and drink sample, and a liquid sample based thereon, or a diluted or concentrated liquid based thereon containing an increased or decreased amount of water.

### <Detection Reagent>

The detection reagent that reacts with the certain functional group preferably reacts with the measurement object to emit a signal, and preferably shows, for example, color development, color change, fluorescence, or luminescence. The reaction between the certain functional group and the detection reagent is preferably an organic reaction.

Examples of the detection reagent that reacts with the certain functional group include detection reagents each of which reacts with a thiol group, an amino group, a carboxy group, a carbonyl group, a hydroxy group, or the like. All kinds of publicly known reagents are known as such detection reagents. Examples of the detection reagent that reacts with an amino group may include detection reagents having isocyanate, sulfonic acid chloride, carbodiimide, and N-hydroxysuccinimide structures, examples of the detection reagent that reacts with a carboxy group may include compounds having a haloacetyl group and a haloalkyl group, and an example of the detection reagent that reacts with a hydroxy group may be 3,5-dinitrobenzoyl chloride.

Examples of the detection reagent that reacts with a thiol group may include: a compound that causes a reaction, such as a nucleophilic substitution reaction, a Michael addition reaction, a thiol-ene reaction, or a disulfide exchange reaction, with a thiol group; and a compound that causes a reaction in which an aldehyde is converted into thiazolidine or thiazinane.

However, the detection reagent is not limited to the foregoing, and any detection reagent capable of reacting with a thiol compound to emit a signal or to make a change may be used. Such compound that reacts with a thiol compound may be a compound having, as a reactive group, haloacetyl, maleimide, aziridine, acryloyl, cyanoacryloyl, an alkylating agent, dinitrophenyl sulfone, vinyl sulfone, pyridyl disulfide, TNB thiol, a disulfide, benzaldehyde, azo, or the like. Alternatively, the compound that reacts with a thiol compound may be a metal complex.

As an especially preferred example of the detection reagent that reacts with a thiol group, there may be given a detection reagent having a monovalent group represented by the following formula (1).

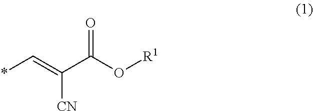

In the formula (1), R¹ represents a hydrogen atom or an alkyl group having 1 or more and 3 or less carbon atoms, and * represents a bonding site.

An example of the detection reagent having a monovalent group may be a compound represented by the following formula (2).

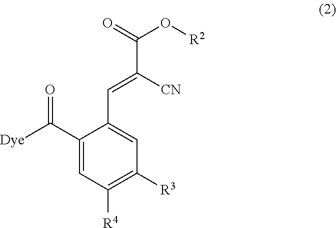

In the formula (2), R² represents a hydrogen atom or an alkyl group having 1 or more and 3 or less carbon atoms, R³ and R⁴ each independently represent a hydrogen atom or a methoxy group, and Dye represents a fluorescent dye having a light absorption band in a wavelength region of from 350 nm to 550 nm.

Preferred specific examples of Dye in the formula (2) are shown in the following formulae (3) to (12). However, Dye is not limited to the following examples. ** represents the position of the C of the C═O of the formula (2). In other words, it may be said that ** represents a bonding site to be bonded to the C of the C═O in the formula (2).

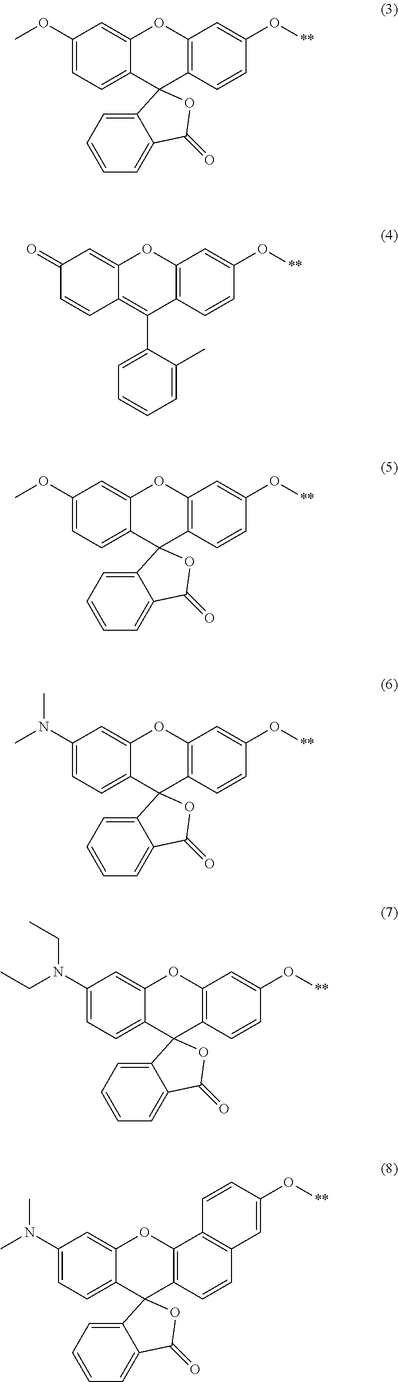

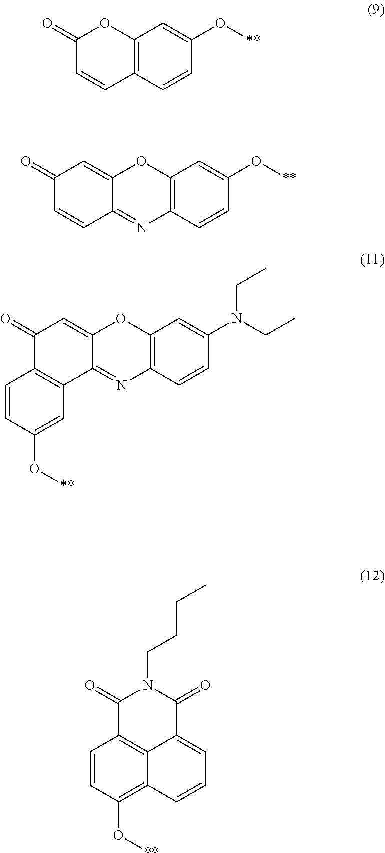

As specific examples of the detection reagent that reacts with a thiol group, there are given detection reagents A to D represented by the following formulae (13) to (16).

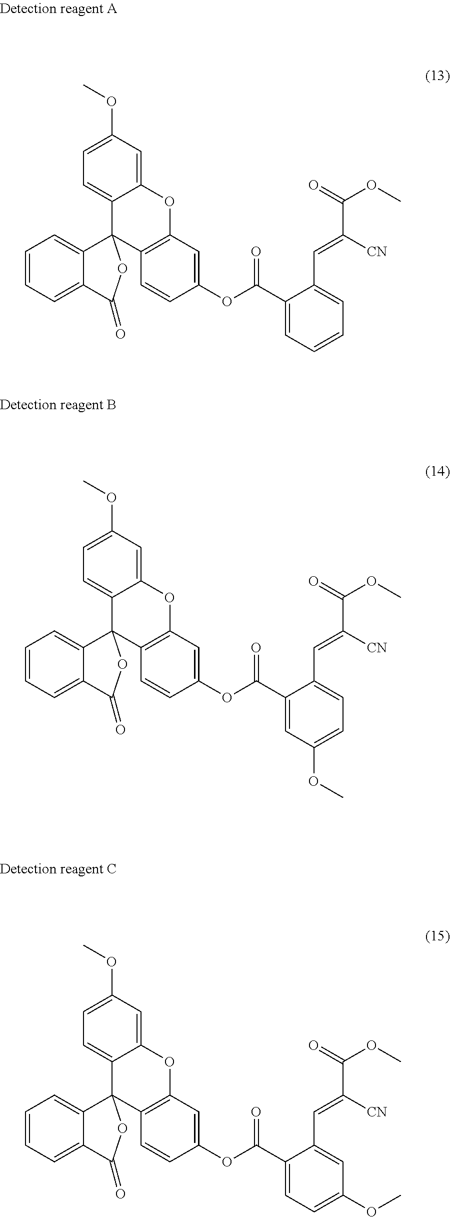 [SMILES: COC(=O)/C(C#N)=C/c1ccccc1C(=O)Oc1ccc2c(c1)Oc1cc(OC)ccc1C21OC(=O)c2ccccc21 ; COC(=O)/C(C#N)=C/c1ccc(OC)cc1C(=O)Oc1ccc2c(c1)Oc1cc(OC)ccc1C21OC(=O)c2ccccc21 ; COC(=O)/C(C#N)=C/c1cc(OC)ccc1C(=O)Oc1ccc2c(c1)Oc1cc(OC)ccc1C21OC(=O)c2ccccc21]

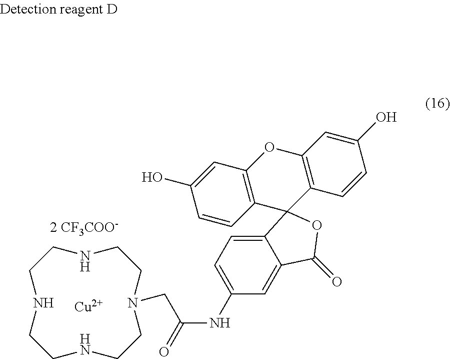

As a plurality of detection reagents, when detection reagents each of which distinguishes between a plurality of measurement objects with high specificity to react with only one kind thereof are available, it is efficient to use such reagents. However, reagents that react with high specificity with compounds having the same functional group are difficult to obtain in many cases. Of those, cysteine, homocysteine, glutathione, and methanethiol, which have an alkylthiol group in common, have extremely high similarities, and hence reagents that react therewith respectively with high specificity are particularly difficult to obtain. Meanwhile, detection reagents that react with the same functional group but have slightly different reactivities with each of the measurement objects are easy to obtain. For example, detection reagents that react with all of cysteine, homocysteine, glutathione, methanethiol, hydrogen sulfide, and the like, but have slightly different reactivities are obtainable. A group of derivatives having slightly different reactivities may be obtained by introducing a substituent in the vicinity of the reactive atom of a detection reagent that reacts with a thiol group, or performing a modification such as atomic substitution. Each of such detection reagents does not identify a measurement object or determine the concentration thereof, but each of the detection reagents emits a different signal for one specimen. Accordingly, the measurement objects can be determined by allowing each of a plurality of kinds of detection reagents to act on one specimen individually, and analyzing the response pattern of the signals obtained from the respective detection reagents. In this embodiment, as described above, detection reagents that react with measurement objects having different structures to emit different signals are obtained, and a plurality thereof are used, which enables information about a plurality of measurement objects to be obtained. That is, in this embodiment, unlike a related-art case in which a certain measurement object is determined through use of one kind of reagent that reacts therewith with high specificity, the measurement objects are determined through utilization of a signal pattern formed of a plurality of signals obtained using a plurality of detection reagents having low specificity but having different reactivities.

For example, when there are a first measurement object and a second measurement object having the same functional group, such a detection reagent X1 that a signal emitted through its reaction with the first measurement object is larger than a signal emitted through its reaction with the second measurement object may be combined with such a detection reagent X2 that, similarly, a signal emitted through its reaction with the second measurement object is larger than a signal emitted through its reaction with the first measurement object. Such combination is useful because, for example, when the specimen contains both of the first measurement object and the second measurement object, and the concentrations of the first measurement object and the second measurement object differ from each other, information on each of the first measurement object and the second measurement object is obtained by comparing the signal from the detection reagent X1 and the signal from the detection reagent X2.

### <Detection Units>

The analysis apparatus according to this embodiment includes a plurality of detection units. The addition unit communicates downstream thereof to the plurality of flow paths, and the plurality of flow paths respectively communicate to the detection units.

Each of the plurality of detection units includes at least one kind of detection reagent that reacts with the certain functional group.

One detection unit may include a plurality of kinds of detection reagents. At least two of the plurality of detection units include detection reagents different from each other. In addition, the plurality of detection units may include detection reagents at concentrations different from each other. Each detection unit may emit a signal through a reaction between the detection reagent and the measurement object.

One detection unit is unlike a related-art one configured to identify the measurement object having the certain functional group or individually determine the concentration thereof, but is used for acquiring signal information emitted from the detection reagent included in the detection unit, and is a signal generation unit for obtaining one signal for forming a signal pattern. The signal pattern is obtained from arrangement information on the plurality of detection units and the signal information obtained from each detection unit, and the measurement object can be determined from such signal pattern.

Each of the plurality of detection units may include, in addition to the detection reagent, an enzyme for producing a compound having the certain functional group. Each detection unit may be capable of emitting a signal even for a measurement object that does not have the certain functional group in the following manner: a compound having the certain functional group is produced from the measurement object through a reaction between the measurement object and the enzyme, and the compound reacts with the detection reagent.

In addition, each detection unit may include an enzyme that uses the measurement object having the certain functional group as a substrate. The concentration of the measurement object can be changed by producing a compound different in reactivity or having no reactivity through a reaction between the measurement object and such enzyme. In this manner, information about a plurality of measurement objects can also be obtained with one detection reagent by obtaining a signal emitted from a detection unit including only the detection reagent and a signal emitted from another detection unit including the detection reagent and the enzyme.

In addition, an enzymatic reaction may be performed in a detection unit including the enzyme, or the specimen may be allowed to react with the enzyme in advance before being added to the analysis apparatus.

A case in which the analysis apparatus according to this embodiment includes “n” kinds of detection units Y1 to Yn having “m” kinds of detection reagents X1 to Xm arranged therein in various concentrations or combinations is assumed. Signal information obtained or assumed when “p” kinds of standard samples (referred to as St1 to Stp) containing measurement objects whose types and concentrations are known in various combinations are added to the analysis apparatus including the detection units Y1 to Yn is obtained in advance. In that case, information on the measurement object can be obtained through comparison between signal information obtained by adding the specimen of interest and the signal information on the standard samples. The signal information may be intensity data on the signal of each of the detection units, or may be the presence or absence of the signal (pattern data). For example, information on the measurement object in the specimen can be determined by obtaining the signal of each of Y1 to Yn at the time of the addition of St1, the signal of each of Y1 to Yn at the time of the addition of St2, the signal of each of Y1 to Yn at the time of the addition of Stp in advance, and comparing those pieces of information to the signal of each of Y1 to Yn at the time of the addition of the specimen.

In this embodiment, the number of the detection units arranged is 2 or more, and there is no particular upper limit. When the signal information is to be recognized through visual observation, the number of the detection units is preferably 2 or more and 12 or less. When an information acquisition unit is arranged, there is no upper limit. However, when the detection units are arranged in a fluidic device, in view of its structure, the number of the detection units is 1,000 or less.

### <Signal Information>

The signal is, for example, a signal selected from the group consisting of: electrical conductivity or resistance; current; potential; capacitance; absorbance; light transmittance; refractive index; fluorescence; phosphorescence; luminescence; color development; color change; and heat quantity measured by calorimetry. The signal may be the color development pattern or color development intensity of each detection unit, or the fluorescence pattern or fluorescence intensity of the detection unit obtained by irradiation with a UV ray or visible light through use of a UV-ray or LED light.

The signal information is information on the signals of the plurality of detection units, and may include at least any one of the presence or absence of the signal of each of the plurality of detection units and the intensity of the signal of each of the plurality of detection units.

In addition, the signal information may include one or more of a hue, a lightness, a fluorescence intensity, a fluorescence wavelength, a luminescence intensity, and a luminescence wavelength of each of the plurality of detection units.

To acquire the signal information from the detection units, an individual information acquisition unit may be arranged for each individual detection unit, or the signals of the plurality of detection units may be acquired by one information acquisition unit. In that case, the plurality of detection units may be arranged within a certain range so that their signals may be simultaneously acquired with one information acquisition unit. The information acquisition unit is preferably capable of setting a coordinate position at which measurement is performed within a measurement range.

The signal information from the detection units is recognized through visual observation, and the type or presence amount of the measurement object may be determined therefrom. Alternatively, the information acquisition unit acquires the signal information, from which a person may determine the type or presence amount of the measurement object. Alternatively, information on the measurement object may be generated by an information processing unit based on the signal information. The recognition through visual observation refers to a determination involving the use of the human eye in any one step, and encompasses: visually observing light emitted using a UV-ray light, an LED light, a reagent, or the like; visually observing an image taken on a smartphone or a camera; performing a comparison to a color sample; and the like.

The information acquisition unit is not particularly limited as long as the information acquisition unit is configured to acquire any of the signals listed above. The information acquisition unit encompasses an irradiation device, such as a laser light or an LED light, a photographing device such as a camera, a smartphone, an absorption photometer, an ammeter, a temperature gauge, and the like, and may include an output unit configured to output the acquired signal information to the information processing unit.

### <Fluidic Device>

The “fluidic device” is a product obtained by forming a flow path pattern on a base material. A solid material may be used as the base material, but the base material preferably has low reactivity with the measurement object and the like. For example, glass, a ceramic, a silicone resin, paper using cellulose or a microfiber, felt, a knitted fabric, a non-woven fabric, a porous material, or filter paper is desired as the base material. In view of easy availability, a paper material is preferred.

The base material in this embodiment may be glass or a resin having a flow path pattern formed thereon through use of a fine processing technology or a machining technology, or a paper material having the frame of a flow path pattern drawn thereon by printing with a hydrophobic material. At least one kind selected from the group consisting of: a wax; a crayon; paraffin; SU-8; silicone; a permanent marker; polyacrylic acid; acrylic lacquer; an alkyl ketone dimer; polystyrene; octadecyltrichlorosilane; polydimethylsiloxane; polyacrylate; and a cyclic olefin copolymer may be used as the hydrophobic material in this embodiment, but the hydrophobic material is not limited thereto.

The flow path pattern may be formed by, for example, placing resin particles on a paper material, which has the frame of the flow path pattern drawn thereon by printing, to form a resin particle image through use of a flow path pattern-forming apparatus. When the base material having the flow path pattern formed thereon undergoes a heating process, the resin particles are melted and permeate the base material, and thus a fluidic device having flow paths surrounded by hydrophobic walls may be produced. In the case of a paper material having printed thereon a flow path pattern including an addition unit, separate flow paths, and detection units, when a detection reagent is applied within the frame of each of the detection units after the formation of the flow path pattern, the detection reagent is held within the frame, and hence the measurement object can be detected on the base material with high sensitivity.

In the fluidic device according to this embodiment, the specimen in the addition unit may be developed on the device to be brought into contact with the detection units through utilization of a pump, or the specimen may be developed on the device to be brought into contact with the detection units through utilization of capillarity like a paper chromatograph.

The amount of change in signal intensity of the color intensity, fluorescence intensity, or luminescence intensity of each of the detection units after a lapse of a certain period of time since the specimen is brought into contact with the detection unit portions on the device is observed through visual observation or measured using a measuring instrument, such as a reflection densitometer or a fluorescent spectrodensitometer. Thus, the signal intensity of the detection reagent may be determined.

### <Reduction Unit>

The analysis apparatus according to this embodiment may include a reduction unit, and the reduction unit is preferably arranged to communicate downstream of the addition unit, and to communicate upstream of the plurality of flow paths. With such structure, substances contained in the specimen can be reduced at one site. The reduction unit includes a reducing agent, and reduces the measurement object. Particularly when the measurement object is a thiol compound, the analysis apparatus preferably includes the reduction unit. That is, when the measurement object is a thiol compound, a disulfide is liable to be formed, but can be turned into the thiol compound by being reduced by the reduction unit.

Examples of the reducing agent may include reducing agents, such as thiols and phosphines, but are not limited thereto, and any reducing agent capable of reducing an oxidized thiol may be used. Examples of such thiols may include: alkylthiols, such as 2-mercaptoethanol and 2-mercaptoethylamine; cysteine; and dithiols such as dithiothreitol. A gel carrier having fixed thereto any of those thiols is preferably used. This is because the reducing agent can be prevented from flowing into the detection unit. Examples of such phosphines may include: trialkylphosphines, such as tripropylphosphine, tri-n-butylphosphine, tri-t-butylphosphine, di-t-butylmethylphosphine, di-t-butylneopentylphosphine, and tris(2-carboxyethyl)phosphine; tricycloalkylphosphines, such as tricyclohexylphosphine and tricyclopentylphosphine; triarylphosphines, such as triphenylphosphine, tri(4-chlorophenyl)phosphine, tri(o- or p-tolyl)phosphine, and diphenyl(o-tolyl)phosphine; and tri(alkyl and/or cycloalkyl and/or aryl)phosphines, such as t-butyldiphenylphosphine and cyclohexyldiphenylphosphine. A gel carrier (a gel support) having fixed thereto any of those phosphines may be used for the reduction unit.

The reduction unit and the addition unit may be integrated with each other, that is, the addition unit may include the reducing agent. In addition, with regard to a reduction reaction, the specimen may be allowed to react with a reducing substance to be reduced in advance before being added to the analysis apparatus.

### <Information Processing Unit>

The analysis apparatus according to this embodiment may include: an information acquisition unit configured to acquire signal information that is information about a signal emitted from each of the plurality of detection units; and an information processing unit configured to generate information on the measurement object from the signal information.

The information processing unit can generate information on the measurement object in the specimen from the signal information. The information on the measurement object, which is generated by the information processing unit, may include at least any one of information about a type of at least one kind of measurement object and information about a presence amount of at least one kind of measurement object.

When the information processing unit generates information on the measurement object in the specimen from the signal information, a reference database may be used. The reference database is described.

For example, a case of including detection units Y1 to Yn having detection reagents X1 to Xm arranged therein in various concentrations or combinations is assumed. Signal information obtained or assumed when standard samples (referred to as St1 to Stp) containing measurement objects whose types and concentrations are known in various combinations are added to the analysis apparatus including the detection units Y1 to Yn is collected in advance, and the collected signal information may be used as reference data. The signal information may be intensity data on the signal of each of the detection units, or may be the presence or absence of the signal (pattern data). For example, the standard sample used and the signals of Y1 to Yn at that time are made to correspond to each other like the signal of each of Y1 to Yn at the time of the addition of St1, the signal of each of Y1 to Yn at the time of the addition of St2, the signal of each of Y1 to Yn at the time of the addition of Stp, and the resultant may be used as reference data. Further, the reference data may include a mathematical expression or the like determined from such correspondence.

Alternatively, when the information processing unit generates information on the measurement object in the specimen from the signal information, a learned model may be used. That is, in the information processing unit, the signal information may be input into the learned model to generate information on the measurement object in the specimen. The learned model may be generated through learning about a specimen for learning, in which at least part of information on the measurement object is known, with the signal information serving as an input and the information on the measurement object serving as an output.

The “learned model” is a model obtained by causing an arbitrary machine learning algorithm to undergo training (learning) through use of appropriate teaching data (learning data) in advance. In this embodiment, the teaching data uses the signal information from the plurality of detection units as input data, and uses information including, for example, the type or concentration of the measurement object in the standard samples as output data, and is made up of a paired group thereof. The learned model may be a regression equation obtained through regression analysis, or may be a neural network model of pattern recognition or the like such as deep learning, and the data structure, learning algorithm, and the like of the model are not limited.

### <Information Processing Device>

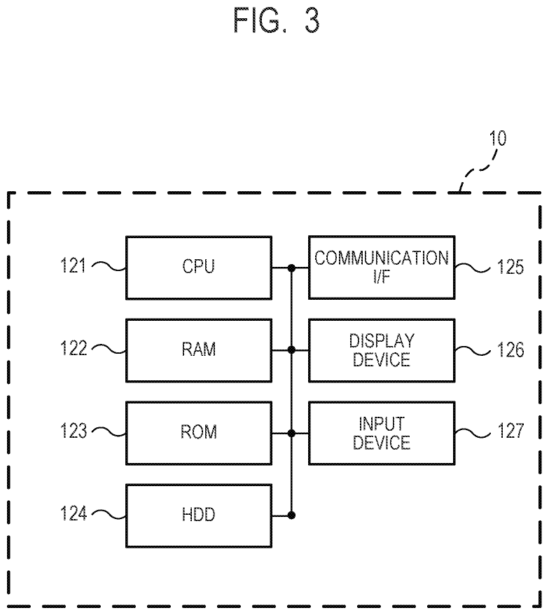

FIG. 3 is a block diagram for illustrating the hardware configuration of an information processing device 10 included in the information processing unit according to this embodiment. The information processing device 10 includes a central processing unit (CPU) 121, a random access memory (RAM) 122, a read only memory (ROM) 123, a hard disk drive (HDD) 124, a communication OF 125, a display device 126, and an input device 127. Those units are connected to each other via a bus or the like.

The CPU 121 is a processor configured to read programs stored in the ROM 123 and the HDD 124 into the RAM 122 to execute the programs, to thereby perform arithmetic processing, control of each unit of the information processing device 10, and the like. The processing performed by the CPU 121 may include: generating information on the measurement object in the specimen through use of a reference database; inputting the signal information into a learned model to generate information on the measurement object in the specimen; and generating a learned model through learning about a specimen for learning, in which at least part of information on the measurement object is known, with the signal information serving as an input and the information on the measurement object serving as an output.

The RAM 122 is a volatile storage medium, and the CPU 121 functions as a work memory in the execution of a program. The ROM 123 is a non-volatile storage medium, and stores firmware required for the operation of the information processing device 10 and the like. The HDD 124 is a non-volatile storage medium, and stores a reference database, a learned model, programs to be used for the acquisition of signal information and the generation of information on an object, and the like.

The communication OF 125 is a communication device based on a standard, such as Wi-Fi (trademark), Ethernet (trademark), or Bluetooth (trademark). The communication OF 125 is used for communication with, for example, the fluidic device, the information acquisition unit, or another computer.

The input device 127 is a device for inputting information into the information processing device 10, and is typically a user interface for allowing a user to operate the information processing device 10. Examples of the input device 127 include a keyboard, a button, a mouse, and a touch panel.

The display device 126 is a device for allowing the information processing device 10 to output information to the outside, and is typically a user interface for presenting information to a user. Examples of the display device 126 include a display and a speaker.

The above-mentioned configuration of the information processing device 10 is merely an example, and may be changed as appropriate. For example, examples of processors that may be installed on the information processing device 10 include a GPU, an ASIC, and an FPGA in addition to the above-mentioned CPU 121. In addition, a plurality of those processors may be arranged, and the plurality of processors may perform processing in a distributed manner. In addition, a function of storing information such as image data in the HDD 124 may be arranged in another data server, not in the information processing device 10. In addition, the HDD 124 may be a storage medium, such as an optical disc, a magneto-optical disk, or a solid state drive (SSD).

The CPU 121 is configured to execute a program to perform predetermined arithmetic processing. In addition, the CPU 121 is configured to execute a program to control each unit in the information processing device 10. Through those kinds of processing, the CPU 121 realizes the functions of the information processing unit.

The hardware configuration illustrated in FIG. 3 is merely illustrative, and as long as the functions of the information processing device 10 according to this embodiment are realized, a device other than those described above may be added, and part of the devices may not be arranged. In addition, part of the devices may be substituted by a different device having a similar function. Further, part of the functions may be provided by another device via a network, and the constituent functions of this embodiment may be realized by being distributed over a plurality of devices. For example, the HDD 124 may be substituted by an SSD using a semiconductor element such as a flash memory.

### <Analysis Kit>

According to a second embodiment of the present invention, there is provided an analysis kit for analyzing a specimen that may contain one or more kinds of measurement objects, the measurement objects each being a substance that satisfies at least any one of the following conditions α) and β): α) having a certain functional group; and β) being capable of reacting with an enzyme to produce a compound having the certain functional group, the analysis kit including a fluidic device including: an addition unit to which the specimen is added; and a plurality of flow paths communicating downstream of the addition unit, and a plurality of detection units respectively communicating to the plurality of flow paths, wherein each of the plurality of detection units includes one kind of detection reagent that reacts with the certain functional group, wherein the detection reagent contains a compound having specific reactivity with the certain functional group, and wherein the plurality of detection units include two or more kinds of detection reagents different from each other in the reactivity.

The analysis kit may include a color sample showing a hue, lightness, or chroma, for showing a relationship between the type or concentration of the measurement object and signal information. The color sample may be printed on a separate body, such as paper or a plastic sheet. The type or concentration of the measurement object contained in the specimen may be determined by comparing information on the signals of the detection units obtained by adding the measurement object to the color sample. In addition, the analysis kit may include an LED light as a separate body. The wavelength of the LED light may be set to fall within the range of from 365 nm to 550 nm. When the detection units are irradiated using the light of the LED light as excitation light, the fluorescence change and fluorescence pattern of the detection units can be seen through visual observation. In addition, the amount or concentration of each measurement object contained in the specimen may be quantitatively determined by acquiring the detection units as image data through use of the camera function of a smartphone, and measuring the color pattern or fluorescence change of the detection units as intensity values through use of an application.

The analysis kit may include a container as a separate body for subjecting the specimen to an enzymatic reaction in advance. When the container is provided as a separate body, the specimen may be added to the container as a separate body and allowed to stay therein for a certain period of time to subject the measurement object to an enzymatic reaction before being added to the fluidic device.

The analysis kit may include a small chamber capable of allowing the specimen and the fluidic device of the present invention to be added or inserted thereinside. The small chamber preferably has an internal structure capable of blocking external light, such as sunlight or a fluorescent lamp. This is because only fluorescence can be selectively detected without being affected by external light. In addition, the small chamber may be integrated with an LED light or a long-pass filter, preferably has a window through which the inside of the small chamber can be seen from the outside through visual observation or through use of a compact digital camera, the camera function of a smartphone, or the like, and preferably has a holder function by which a compact digital camera or a smartphone can be fitted.

### <Analysis Method>

According to a third embodiment of the present invention, there is provided an analysis method for analyzing a specimen that may contain a measurement object, the measurement object being a substance that satisfies at least any one of the following conditions α) and β): α) having a certain functional group; and β) being capable of reacting with an enzyme to produce a compound having the certain functional group, the analysis method including: an introduction step of introducing the specimen into a plurality of detection units via a plurality of flow paths; and a reaction step of allowing the specimen to react with one kind of detection reagent that reacts with the certain functional group in each of the plurality of detection units, the plurality of detection units including two or more kinds of detection reagents that each react with the certain functional group.

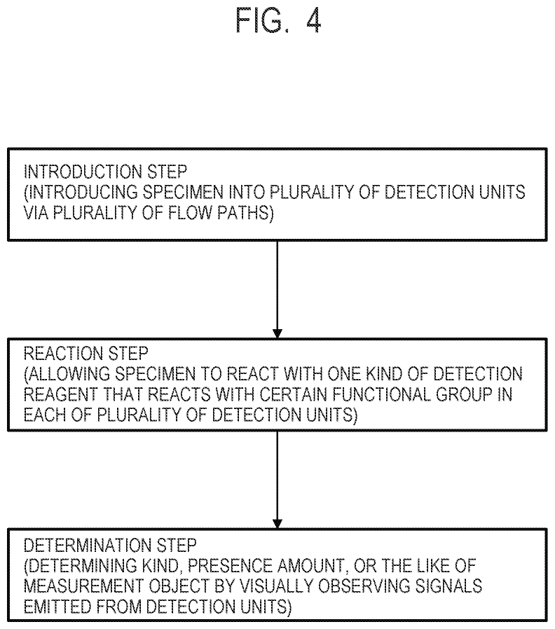

This embodiment is described with reference to FIG. 4. That is, in the introduction step, the specimen is introduced into the plurality of detection units via the plurality of flow paths, and in the reaction step, the specimen is allowed to react with one kind of detection reagent that reacts with the certain functional group in each of the plurality of detection units.

In this embodiment, the analysis method may include, after the reaction step, a step of determining at least any one of a type of at least one kind of the measurement object and a presence amount of at least one kind of the measurement object by visually observing a signal emitted from each of the plurality of detection units.

According to a fourth embodiment of the present invention, there is provided an analysis method for analyzing a specimen that may contain a measurement object, the analysis method including: a reduction step of reducing the specimen by passage through a reduction unit via a flow path; an introduction step of introducing the specimen from the reduction unit into a plurality of detection units via a plurality of flow paths; and a reaction step of allowing the specimen to react with one kind of detection reagent that reacts with the certain functional group in each of the plurality of detection units, the plurality of detection units including two or more kinds of detection reagents that each react with the certain functional group.

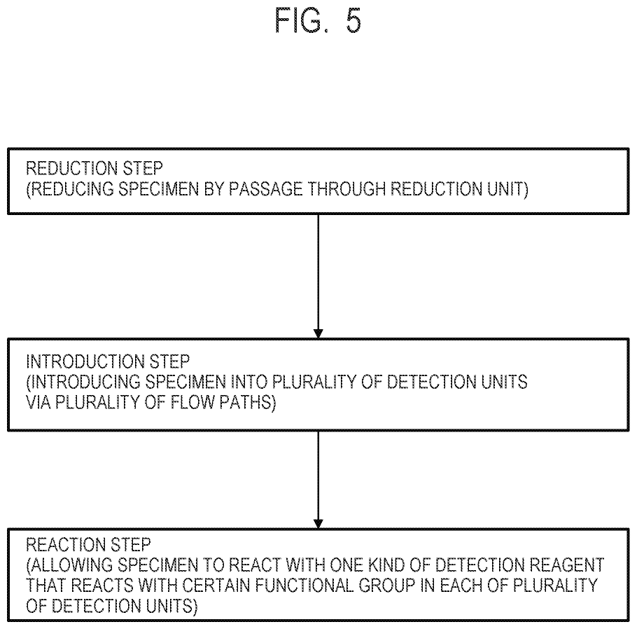

This embodiment is described with reference to FIG. 5. That is, in the reduction step, the specimen is reduced by passage through the reduction unit. In the introduction step, the specimen is introduced into the plurality of detection units via the plurality of flow paths. In the reaction step, the specimen is allowed to react with one kind of detection reagent that reacts with the certain functional group in each of the plurality of detection units.

According to a fifth embodiment of the present invention, there is provided an analysis method for analyzing a specimen that may contain a measurement object, the measurement object being a substance that satisfies at least any one of the following conditions α) and β): α) having a certain functional group; and β) being capable of reacting with an enzyme to produce a compound having the certain functional group, the analysis method including: an introduction step of introducing the specimen into a plurality of detection units via a plurality of flow paths; a reaction step of allowing the specimen to react with one kind of detection reagent that reacts with the certain functional group in each of the plurality of detection units; an information acquisition step of acquiring signal information that is information about a signal emitted from each of the plurality of detection units; and an information processing step of generating information on the measurement object from the signal information, the plurality of detection units including two or more kinds of detection reagents that each react with the certain functional group.

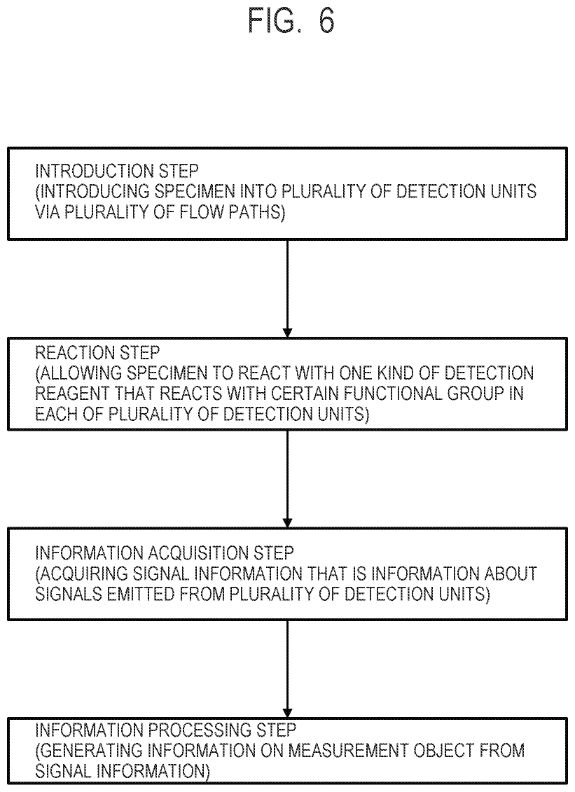

This embodiment is described with reference to FIG. 6. That is, in the introduction step, the specimen is introduced into the plurality of detection units via the plurality of flow paths. In the reaction step, the specimen is allowed to react with one kind of detection reagent that reacts with the certain functional group in each of the plurality of detection units. In the information acquisition step, signal information that is information about a signal emitted from each of the plurality of detection units is acquired. In the information processing step, information on the measurement object is generated from the signal information.

In the information processing step, the information on the measurement object in the specimen may be generated from the signal information through use of a reference database. Alternatively, in the information processing step, the signal information may be input into a learned model to generate the information on the measurement object in the specimen. The learned model is generated through learning about a specimen for learning, in which at least part of information on the measurement object is known, with the signal information serving as an input and the information on the measurement object serving as an output.

### Example 1

In this Example, resin particles (hereinafter also referred to as “COC particles”) produced from a cyclic olefin copolymer (manufactured by Polyplastics Co., Ltd., TM grade, hereinafter also referred to as “COC”) obtained by copolymerizing ethylene and a cyclic olefin were used as a flow path-forming agent.

The cyclic olefin copolymer has two kinds of units (an ethylene unit and a norbornene unit) represented by the following formula (1-1). A content ratio between “x” and “y” in the formula is 85:15 in terms of a molar ratio. The formula (1-1) represents the bonding of the two kinds of units to each other at a predetermined ratio, and does not mean that the cyclic olefin copolymer has a block polymer-like configuration in which a polyethylene moiety and a polynorbornene moiety are bonded to each other.

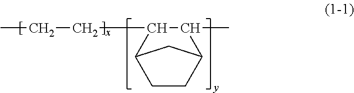

### <Flow Path Pattern>

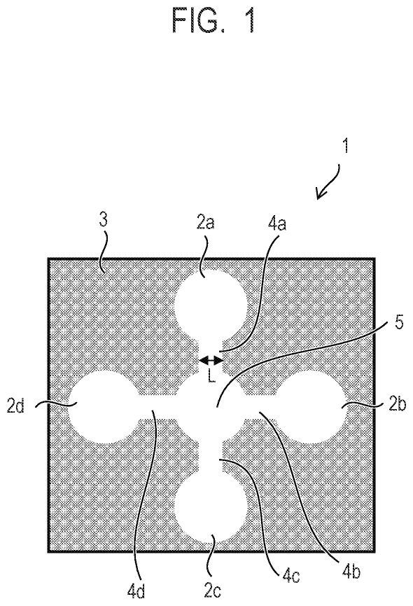

In this Example, a fluidic device 1 illustrated in FIG. 1 was formed by placing COC particles A on a porous base material to form an image of the COC particles A through use of a flow path pattern-forming apparatus. In FIG. 1, the fluidic device 1 included: detection units 2a, 2b, 2c, and 2d including detection reagents; a COC resin particle image 3; an addition unit 5 configured to introduce a specimen; and flow paths 4a, 4b, 4c, and 4d configured to connect the addition unit 5 to the detection units 2a, 2b, 2c, and 2d, respectively. A width L of each of the flow paths 4a, 4b, 4c, and 4d was 1.5 mm. However, the shape, size, and the like of the flow path pattern are merely an example, and the flow path pattern is not limited thereto. A shape that is a combination of a straight line and a curve, or uses a junction may be adopted, and the width of each of the flow paths may be changed.

An analysis apparatus is obtained by using the fluidic device of FIG. 1, and applying, as a detection reagent that reacts with a functional group, the detection reagent A represented by the formula (13) to the detection unit 2a, the detection reagent B represented by the formula (14) to the detection unit 2b, the detection reagent C represented by the formula (15) to the detection unit 2c, and the detection reagent D represented by the formula (16) to the detection unit 2d. When a specimen containing a thiol compound is added to the addition unit 5, the added specimen is separated and diffused to the detection units 2a, 2b, 2c, and 2d through the flow paths 4a, 4b, 4c, and 4d. The specimen reacts only with the detection reagent A in the detection unit 2a, reacts only with the detection reagent B in the detection unit 2b, reacts only with the detection reagent C in the detection unit 2c, and reacts only with the detection reagent D in the detection unit 2d. The use of the separated flow paths enables reactions with a plurality of detection reagents to be simultaneously tested. It is appropriate that a measurement apparatus or a measurer recognize, for example, a coloring reaction or fluorescence reaction resulting at that time.

<Detection Reagents that React with Functional Group>

The detection reagents that react with a functional group (thiol detection reagents) are suitably reagents that have different reactivities with thiol compounds having different structures, and that show changes in fluorescence intensity or color development amount or differences in absorption wavelength or fluorescence wavelength in correspondence with the reactivities. Such reagents that, as the reactivity becomes higher, the color development amount or the fluorescence intensity increases or the absorption wavelength or the fluorescence wavelength changes more significantly are preferred. In addition, as a combination of thiol detection reagents, for example, it is suitable to combine a thiol detection reagent having the highest reactivity with cysteine, a thiol detection reagent having the highest reactivity with homocysteine, a thiol detection reagent having the highest reactivity with glutathione, and a thiol detection reagent showing the highest reactivity with a hydrogen sulfide ion. However, the kinds of thiol detection reagents having the highest reactivities are not necessarily required to differ from each other, and for example, thiol detection reagents that each have the highest reactivity with cysteine but differ in difference in reactivity with other thiol compounds, that is, differ in reaction selectivity or reaction degree, may be used in combination.

### <Identification of Compounds>

Compounds synthesized below were each identified with a ¹H-NMR measurement device (manufactured by Bruker, Bruker Avance 500, resonant frequency: 500 MHz).

### (Production Example 1) Synthesis of Detection Reagent A

64 mg of a white solid was obtained with reference to Org Lett. 2012, 14, 2184-2187.

### (Production Example 2) Synthesis of Detection Reagent B

The detection reagent B was obtained through the following steps. The details of each step are described below.

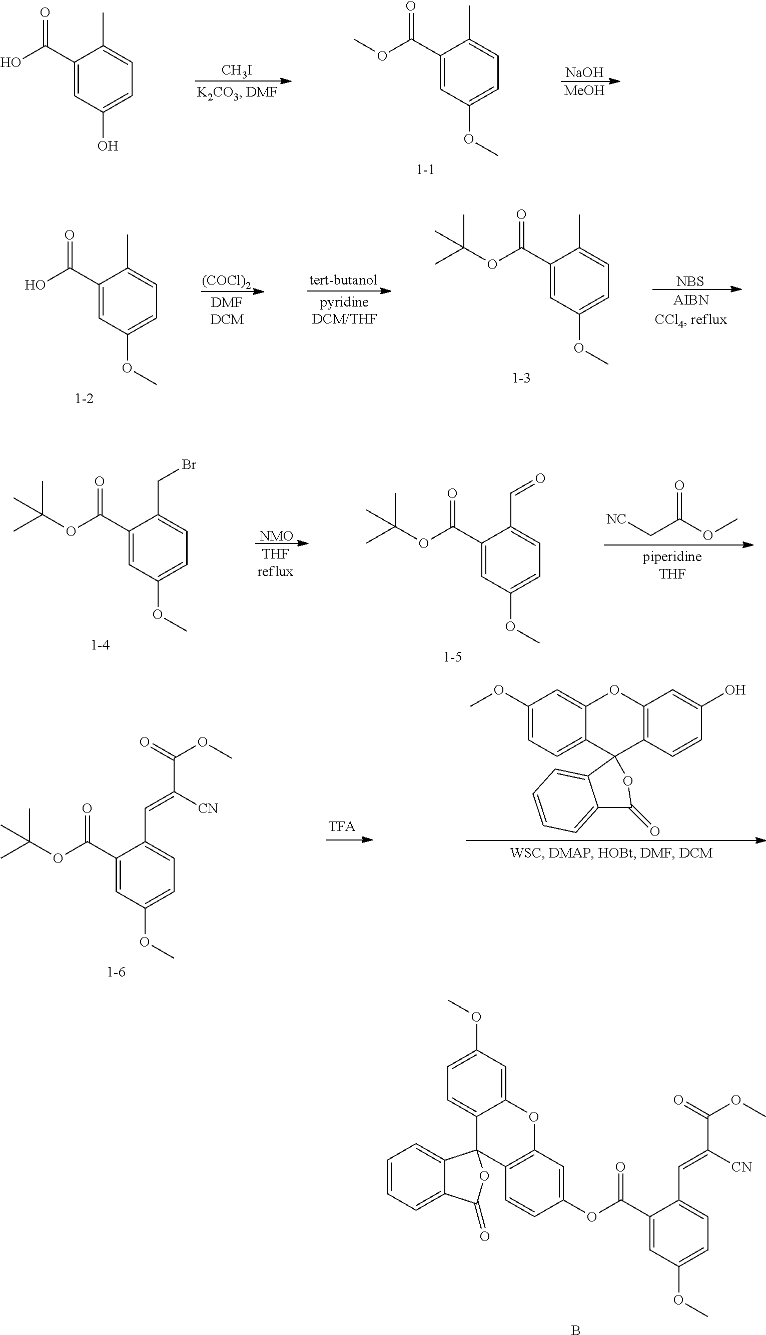

### (Step 1) Synthesis of Compound 1-1

8.2 g of 5-hydroxy-2-methylbenzoic acid, 37 g of potassium carbonate, and 46 g of methyl iodide were added to 20 mL of N,N-dimethylformamide, and the whole was stirred at 70° C. overnight. After the resultant had been returned to room temperature, 150 mL of water was added thereto in an ice bath, and the mixture was stirred at room temperature for 20 minutes. 120 mL of ethyl acetate and 30 mL of heptane were added, and the whole was washed with water twice. Further washing was performed with saturated sodium bicarbonate water and brine. The organic layer was collected, dehydrated by adding sodium sulfate, and then filtered. The solvent of the filtrate was removed by evaporation to give 9.8 g of a yellow oil (yield: 98%). ¹H-NMR (CDCl₃) (ppm): 7.44 (d, J=2.5 Hz, 1H), 7.14 (d, J=8.5 Hz, 1H), 6.96 (dd, J=8.5, 2.5 Hz, 1H), 3.89 (s, 3H), 3.82 (s, 3H), 2.52 (s, 3H).

### (Step 2) Synthesis of Compound 1-2

9.8 g of Compound 1-1 and 50 mL of a 3M aqueous solution of sodium hydroxide were added to 100 mL of methanol, and the whole was stirred at 60° C. overnight. After the resultant had been returned to room temperature and the solvent had been removed by evaporation, 100 mL of water was added, and concentrated hydrochloric acid was added dropwise to the mixture in an ice bath to adjust its pH to 7. 120 mL of chloroform was added for extraction. The chloroform layer was collected, dehydrated by adding sodium sulfate, and then filtered. The solvent of the filtrate was removed by evaporation to give 7.7 g of a colorless oil (yield: 88%). ¹H-NMR (CDCl₃) (ppm): 11.71 (br, 1H), 7.59 (d, J=2.5 Hz, 1H), 7.18 (d, J=8.5 Hz, 1H), 7.02 (dd, J=8.5, 2.5 Hz, 1H), 3.84 (s, 3H), 2.58 (s, 3H). (Step 3) Synthesis of Compound 1-3

2.6 g of Compound 1-2 and 0.5 mL of N,N-dimethylformamide were added to 30 mL of dichloromethane, and the whole was stirred in an ice bath for 10 minutes. 20 mL of dichloromethane containing 1.8 mL of oxalyl chloride was added dropwise. After the resultant had been returned to room temperature and stirred overnight, the solvent was removed by evaporation. 20 mL of tetrahydrofuran containing 3.5 mL of tert-butanol and 3.5 mL of pyridine was added dropwise to the residue in an ice bath, and then the whole was heated to reflux under a stream of nitrogen overnight. After the resultant had been returned to room temperature, 50 mL of water was added, and the whole was stirred for 20 minutes. Chloroform was added, and the whole was washed with saturated sodium bicarbonate water, a saturated aqueous solution of ammonium chloride, and brine. The chloroform layer was collected, dehydrated by adding sodium sulfate, and then filtered. The solvent of the filtrate was removed by evaporation, and then the residue was purified by silica gel column chromatography using ethyl acetate/heptane (1/10) as an eluting solvent to give 1.2 g of a colorless oil (yield: 35%). ¹H-NMR (CDCl₃) (ppm): 7.36 (d, J=3.0 Hz, 1H), 7.11 (d, J=8.0 Hz, 1H), 6.92 (dd, J=8.0, 3.0 Hz, 1H), 3.81 (s, 3H), 2.48 (s, 3H), 1.59 (s, 9H).

### (Step 4) Synthesis of Compound 1-4

0.42 g of Compound 1-3, 0.37 g of N-bromosuccinimide (NBS), and 0.015 g of azobisisobutyronitrile were added to 4 mL of carbon tetrachloride, and the whole was heated to reflux under a stream of nitrogen for 3 hours. After the resultant had been returned to room temperature, 40 mL of water was added thereto in an ice bath, and the mixture was stirred for 5 minutes. Chloroform was added for extraction. The chloroform layer was collected, dehydrated by adding sodium sulfate, and then filtered. The solvent of the filtrate was removed by evaporation, and then the residue was purified by silica gel column chromatography using ethyl acetate/heptane (1/10) as an eluting solvent to give 0.41 g of a colorless oil (yield: 73%). ¹H-NMR (CDCl₃) (ppm): 7.41 (d, J=3.0 Hz, 1H), 7.33 (d, J=8.5 Hz, 1H), 6.97 (dd, J=8.5, 3.0 Hz, 1H), 4.89 (s, 2H), 3.84 (s, 3H), 1.64 (s, 9H).

### (Step 5) Synthesis of Compound 1-5

0.41 g of Compound 1-4 and 0.50 g of N-methylmorpholine N-oxide (NMO) were added to 50 mL of tetrahydrofuran in an ice bath, and the mixture was heated to reflux under a stream of nitrogen for 3 hours. After the resultant had been returned to room temperature, 50 mL of water was added thereto in an ice bath, and the mixture was stirred for 5 minutes. Chloroform was added for extraction. The chloroform layer was collected, dehydrated by adding sodium sulfate, and then filtered. The solvent of the filtrate was removed by evaporation, and then the residue was purified by silica gel column chromatography using ethyl acetate/heptane (1/5) as an eluting solvent to give 0.30 g of a colorless oil (yield: 93%). ¹H-NMR (CDCl₃) (ppm): 10.50 (s, 1H), 7.93 (d, J=8.5 Hz, 1H), 7.32 (d, J=2.5 Hz, 1H), 7.08 (dd, J=8.5, 2.5 Hz, 1H), 3.91 (s, 3H), 1.62 (s, 9H).

### (Step 6) Synthesis of Compound 1-6

0.18 g of Compound 1-5, 0.12 g of methyl cyanoacetate, and 0.3 mL of piperidine were added to 10 mL of tetrahydrofuran, and the whole was stirred at room temperature for 5 hours. The solvent was removed by evaporation, and then the residue was purified by silica gel column chromatography using ethyl acetate/heptane (1/3) as an eluting solvent to give 0.25 g of a colorless oil. ¹H-NMR (CDCl₃) (ppm): 8.93 (s, 1H), 8.00 (d, J=8.5 Hz, 1H), 7.52 (d, J=3.0 Hz, 1H), 7.12 (dd, J=8.5, 3.0 Hz, 1H), 3.93 (s, 3H), 3.91 (s, 3H), 1.60 (s, 9H).

### (Step 7) Synthesis of Compound B

2 mL of trifluoroacetic acid was added to 0.24 g of Compound 1-6, and the whole was stirred at room temperature for 6 hours. The solvent was removed by evaporation, and then 20 mg of the residue was redissolved in 2 mL of dichloromethane. To the solution, 30 mg of 1-ethyl-3-[3-(dimethylamino)propyl]carbodiimide and 1 mg of 4-(dimethylamino)pyridine were added, and the whole was stirred under a stream of argon at room temperature for 1 hour. 35 mg of 3′-O-methylfluorescein and 1 mg of 1-hydroxybenzotriazole were added, and the whole was stirred under a stream of argon at room temperature overnight. The solvent was removed by evaporation, and then the residue was purified by silica gel column chromatography using chloroform as an eluting solvent to give 4 mg of a white solid (yield: 9%). ¹H-NMR (CDCl₃) (ppm): 8.99 (s, 1H), 8.05-8.03 (m, 2H), 7.81 (m, 1H), 7.70-7.62 (m, 2H), 7.27-7.25 (m, 1H), 7.22 (m, 1H), 7.20-7.18 (m, 1H), 6.93-6.91 (m, 1H), 6.89-6.87 (m, 1H), 6.80 (m, 1H), 6.73-6.71 (m, 1H), 6.66-6.64 (m, 1H), 3.96 (s, 3H), 3.90 (s, 3H), 3.85 (s, 3H).

### (Production Example 3) Synthesis of Detection Reagent C

The detection reagent C was obtained through the following steps. The details of each step are described below.

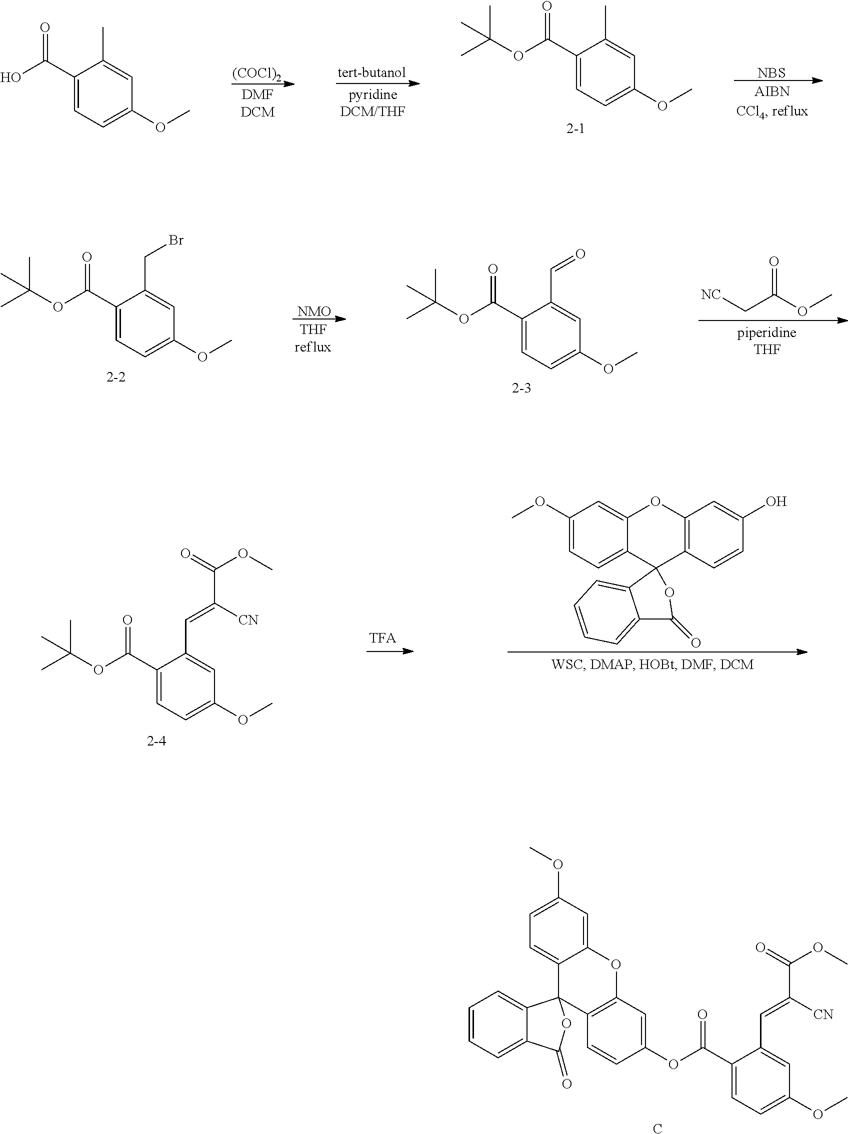

### (Step 1) Synthesis of Compound 2-1

5.3 g of 4-methoxy-2-methylbenzoic acid and 0.5 mL of N,N-dimethylformamide were added to 20 mL of dichloromethane, and the whole was stirred in an ice bath for 10 minutes. 10 mL of dichloromethane containing 2.8 mL of oxalyl chloride was added dropwise. After the resultant had been returned to room temperature and stirred overnight, the solvent was removed by evaporation. To the residue, 40 mL of dichloromethane was added, and the whole was stirred in an ice bath for 10 minutes. 40 mL of tetrahydrofuran containing 8.0 mL of tert-butanol and 8.0 mL of pyridine was added dropwise, and then the whole was heated to reflux under a stream of nitrogen overnight. After the resultant had been returned to room temperature, 50 mL of water and 50 mL of chloroform were added, and the whole was stirred for 10 minutes. Chloroform was added, and the whole was washed with saturated sodium bicarbonate water, a saturated aqueous solution of ammonium chloride, and brine. The chloroform layer was collected, dehydrated by adding sodium sulfate, and then filtered. The solvent of the filtrate was removed by evaporation, and then the residue was purified by silica gel column chromatography using ethyl acetate/heptane (1/10) as an eluting solvent to give 5.7 g of a colorless oil (yield: 81%). ¹H-NMR (CDCl₃) (ppm): 7.85 (m, 1H), 6.74-6.71 (m, 2H), 6.92 (dd, J=8.0, 3.0 Hz, 1H), 3.82 (s, 3H), 2.57 (s, 3H), 1.58 (s, 9H).

### (Step 2) Synthesis of Compound 2-2

2.0 g of Compound 2-1, 1.8 g of N-bromosuccinimide (NBS), and 0.074 g of azobisisobutyronitrile were added to 15 mL of carbon tetrachloride, and the mixture was heated to reflux under a stream of nitrogen for 3 hours. After the resultant had been returned to room temperature, 30 mL of water was added thereto in an ice bath, and the mixture was stirred for 5 minutes. Chloroform was added for extraction. The chloroform layer was collected, dehydrated by adding sodium sulfate, and then filtered. The solvent of the filtrate was removed by evaporation, and then the residue was purified by silica gel column chromatography using ethyl acetate/heptane (1/10) as an eluting solvent to give 1.4 g of a colorless oil (yield: 52%). ¹H-NMR (CDCl₃) (ppm): 7.90 (d, J=8.5 Hz, 1H), 6.93 (d, J=2.5 Hz, 1H), 6.84 (dd, J=8.5, 2.5 Hz, 1H), 4.92 (s, 2H), 3.85 (s, 3H), 1.62 (s, 9H).

### (Step 3) Synthesis of Compound 2-3

1.4 g of Compound 2-2 and 2.2 g of N-methylmorpholine N-oxide (NMO) were added to 10 mL of tetrahydrofuran in an ice bath, and the mixture was heated to reflux under a stream of nitrogen for 2.5 hours. After the resultant had been returned to room temperature, 50 mL of water was added thereto in an ice bath, and the mixture was stirred for 5 minutes. Chloroform was added for extraction. The chloroform layer was collected, dehydrated by adding magnesium sulfate, and then filtered. The solvent of the filtrate was removed by evaporation, and then the residue was purified by silica gel column chromatography using ethyl acetate/heptane (1/5) as an eluting solvent to give 0.89 g of a colorless oil (yield: 80%). ¹H-NMR (CDCl₃) (ppm): 10.67 (s, 1H), 7.91 (d, J=8.5 Hz, 1H), 7.36 (d, J=2.5 Hz, 1H), 7.09 (dd, J=8.5, 2.5 Hz, 1H), 3.89 (s, 3H), 1.61 (s, 9H).

### (Step 4) Synthesis of Compound 2-4

0.51 g of Compound 2-3, 0.34 g of methyl cyanoacetate, and 0.5 mL of piperidine were added to 10 mL of tetrahydrofuran, and the whole was stirred at room temperature overnight. The solvent was removed by evaporation, and then the residue was purified by silica gel column chromatography using ethyl acetate/heptane (1/3) as an eluting solvent to give 0.41 g of a colorless oil (yield: 60%). ¹H-NMR (CDCl₃) (ppm): 9.01 (s, 1H), 8.02 (d, J=8.5 Hz, 1H), 7.28 (d, J=2.5 Hz, 1H), 7.03 (dd, J=8.5, 2.5 Hz, 1H), 3.95 (s, 3H), 3.90 (s, 3H), 1.58 (s, 9H).

### (Step 5) Synthesis of Compound C

2 mL of trifluoroacetic acid was added to 0.41 g of Compound 2-4, and the whole was stirred at room temperature for 6 hours. The solvent was removed by evaporation, and then 0.11 g of the residue was redissolved in 10 mL of dichloromethane. To the solution, 0.25 g of 1-ethyl-3-[3-(dimethylamino)propyl]carbodiimide and 3 mg of 4-(dimethylamino)pyridine were added, and the whole was stirred under a stream of argon at room temperature for 1 hour. 0.16 g of 3′-O-methylfluorescein and 33 mg of 1-hydroxybenzotriazole were added, and the whole was stirred under a stream of argon at room temperature overnight. The solvent was removed by evaporation, and then the residue was purified by silica gel column chromatography using chloroform as an eluting solvent to give 14 mg of a white solid (yield: 6%). ¹H-NMR (CDCl₃) (ppm): 9.03 (s, 1H), 8.31 (d, J=8.5 Hz, 1H), 8.04 (m, 1H), 7.70-7.62 (m, 2H), 7.34 (d, J=2.5 Hz, 1H), 7.19-7.17 (m, 2H), 7.13 (dd, J=8.5, 2.5 Hz, 1H), 6.90-6.88 (m, 1H), 6.87-6.85 (m, 1H), 6.79 (m, 1H), 6.72-6.71 (m, 1H), 6.65-6.63 (m, 1H), 3.96 (s, 3H), 3.91 (s, 3H), 3.85 (s, 3H).

### <Analysis>

Analysis of thiol compounds was performed using a device including detection units formed by separately applying 0.4 nmoL each of the detection reagents A, B, C, and D as thiol detection reagents.

A 200 μM aqueous solution of cysteine was added to the addition unit, and after a lapse of 30 minutes, irradiation with UV light was performed to acquire fluorescence images of A, B, C, and D. Similarly, fluorescence images were also acquired for an aqueous solution containing no thiol compound by a similar operation.

Through use of Image J software, fluorescence emitted from each of A, B, C, and D was digitized, and the change ratio of a result obtained by dropping the aqueous solution of cysteine with respect to a result obtained by dropping the aqueous solution containing no thiol compound was calculated. The fluorescence change ratios of A, B, C, and D were 2.30, 2.20, 3.94, and 1.06, respectively.

A 200 μM aqueous solution of homocysteine was added, and an experiment was performed in the same manner as described above. The fluorescence change ratios of A, B, C, and D were 2.60, 4.02, 5.08, and 1.13, respectively.

A 200 μM aqueous solution of glutathione (reduced form) was added, and an experiment was performed in the same manner. The fluorescence change ratios of A, B, C, and D were 2.28, 3.22, 3.99, and 1.08, respectively.

A 200 μM aqueous solution of sodium sulfide was added, and an experiment was performed in the same manner. The fluorescence change ratios of A, B, C, and D were 1.57, 1.64, 2.01, and 8.03, respectively.

The results of the fluorescence change ratios of A, B, C, and D for each of the thiol compounds are shown in Table 1.

TABLE 1

| Thiol | Fluorescence | Fluorescence | Fluorescence | Fluorescence |
|---|---|---|---|---|
| compound | change ratio | change ratio | change ratio | change ratio |
| in solution | of A | of B | of C | of D |
| Cysteine | 2.30 | 2.20 | 3.94 | 1.06 |
| Homocysteine | 2.60 | 4.02 | 5.08 | 1.13 |
| Glutathione | 2.28 | 3.22 | 3.99 | 1.08 |
| (reduced | | | | |
| form) | | | | |
| Sodium sulfide | 1.57 | 1.64 | 2.01 | 8.03 |

### <Signal Pattern of Fluidic Device>

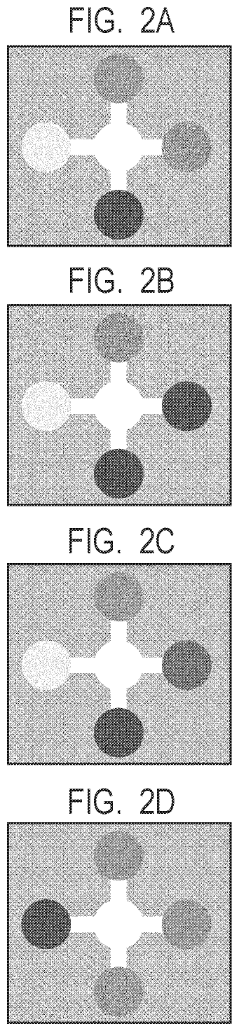

In the case of using the above-mentioned fluidic device, the image patterns of fluorescence intensities or fluorescence changes observed from the detection units of the fluidic device at the time of the addition of the respective thiol compounds are as shown in FIG. 2A, FIG. 2B, FIG. 2C, and FIG. 2D based on the results of Table 1. The case of adding cysteine is as shown in FIG. 2A, the case of homocysteine is as shown in FIG. 2B, the case of glutathione (reduced form) is as shown in FIG. 2C, and the case of sodium sulfide or the hydrogen sulfide ion is as shown in FIG. 2D.

Thiol compounds contained in an unknown specimen can be identified and quantified by comparing a signal pattern emitted from the detection units through addition of the unknown specimen in similarities to the image patterns of FIG. 2A, FIG. 2B, FIG. 2C, and FIG. 2D, and calculating fluorescence intensity values and fluorescence intensity ratios between the detection units, followed by comparison to Table 1.

As described above, it was found that, when the fluidic device according to Examples of the present invention was used, the thiol compounds were able to be determined without performing component separation.

### Example 2

### <Flow Path Pattern>

In this Example, an analysis apparatus was obtained by using the fluidic device of FIG. 1 as in Example 1, and applying, as a detection reagent that reacts with a functional group, the detection reagent B represented by the formula (14) to the detection unit 2a, the detection reagent C represented by the formula (15) to the detection unit 2b, the detection reagent B and an enzyme to the detection unit 2c, and the detection reagent C and an enzyme to the detection unit 2d. When a specimen containing a thiol compound is added to the addition unit 5, the added specimen is separated and diffused to the detection units 2a, 2b, 2c, and 2d through the flow paths 4a, 4b, 4c, and 4d. The specimen reacts only with the detection reagent B in the detection unit 2a, reacts only with the detection reagent C in the detection unit 2b, reacts with the enzyme and the detection reagent B in the detection unit 2c, and reacts with the enzyme and the detection reagent C in the detection unit 2d. The use of the separated flow paths enables reactions with a plurality of detection reagents to be simultaneously tested. It is appropriate that a measurement apparatus or a measurer recognize, for example, a resulting coloring reaction or fluorescence reaction.

<Detection Reagents that React with Functional Group>

As in Example 1, the detection reagents that react with a functional group (thiol detection reagents) are suitably reagents that have different reactivities with thiol compounds having different structures, and that show changes in fluorescence intensity or color development amount or differences in absorption wavelength or fluorescence wavelength in correspondence to the reactivities. Thiol detection reagents that differ in reaction selectivity or reaction degree may be used in combination.

### <Enzyme>

In this Example, the enzyme is suitably an enzyme that produces from a substrate a compound having different reactivity with the detection reagent that reacts with the functional group (thiol detection reagent). An enzyme that produces a compound that generates a difference in reactivity with the detection reagent as compared to the substrate is preferred, and an enzyme that produces such a compound that the difference is large is more preferred. In addition, the enzyme to be used together with the thiol detection reagent is suitably, for example, methionine y-lyase, which is an enzyme that produces methanethiol, which reacts with the thiol detection reagent, from methionine, which does not react with the thiol detection reagent. The combination thereof with the thiol detection reagent enables the detection of methionine, which is difficult to detect with the thiol detection reagent alone. However, the enzyme does not need to be an enzyme that uses a compound that does not react with the thiol detection reagent as the substrate. Methionine γ-lyase and homocysteine γ-lyase can produce hydrogen sulfide ions from homocysteine and cysteine. For example, a hydrogen sulfide ion may be produced from homocysteine or cysteine to generate a difference in reactivity, to thereby detect homocysteine or cysteine based on the difference between the case of using the enzyme in combination and the case of not using the enzyme in combination. That is, an enzyme that produces a compound having different reactivity may be used in combination with the thiol detection reagent.

### <Analysis>

Analysis of thiol compounds was performed using the device with 0.4 nmoL of the detection reagent B, 0.4 nmoL of the detection reagent C, a mixture B′ of 0.4 nmoL of the detection reagent B and 1.5 μg of methionine γ-lyase, and a mixture C′ of 0.4 nmoL of the detection reagent C and 1.5 μg of methionine γ-lyase separately applied as thiol detection reagents.

A 240 μM aqueous solution of cysteine was added to the addition unit, and after a lapse of 10 minutes, irradiation with UV light was performed to acquire fluorescence images of B, C, B′, and C′. Similarly, fluorescence images were also acquired for an aqueous solution containing no thiol compound by a similar operation. Through use of Image J software, fluorescence emitted from each of B, C, B′, and C′ was digitized, and the change ratio of a result obtained by dropping the aqueous solution of cysteine with respect to a result obtained by dropping the aqueous solution containing no thiol compound was calculated. The fluorescence change ratios of B, C, B′, and C′ were 3.20, 3.50, 2.42, and 2.51, respectively.

A 240 μM aqueous solution of homocysteine was added, and an experiment was performed in the same manner as described above. The fluorescence change ratios of B, C, B′, and C′ were 7.37, 4.74, 3.01, and 5.25, respectively.

A 240 μM aqueous solution of glutathione (reduced form) was added, and an experiment was performed in the same manner. The fluorescence change ratios of B, C, B′, and C′ were 4.09, 5.17, 3.17, and 3.29, respectively.

A 240 μM aqueous solution of methionine was added, and an experiment was performed in the same manner. The fluorescence change ratios of B, C, B′, and C′ were 1.37, 1.74, 2.51, and 1.63, respectively.

The results of the fluorescence change ratios of B, C, B′, and C′ for each of the thiol compounds are shown in Table 2.

TABLE 2

| Thiol | Fluorescence | Fluorescence | Fluorescence | Fluorescence |
|---|---|---|---|---|
| compound | change ratio | change ratio | change ratio | change ratio |
| in solution | of B | of C | of B′ | of C′ |
| Cysteine | 3.20 | 3.50 | 2.42 | 2.51 |
| Homocysteine | 7.37 | 4.74 | 3.01 | 5.25 |
| Glutathione | 4.09 | 5.17 | 3.17 | 3.29 |
| (reduced | | | | |
| form) | | | | |
| Methionine | 1.37 | 1.74 | 2.51 | 1.63 |

### <Signal Pattern of Fluidic Device>

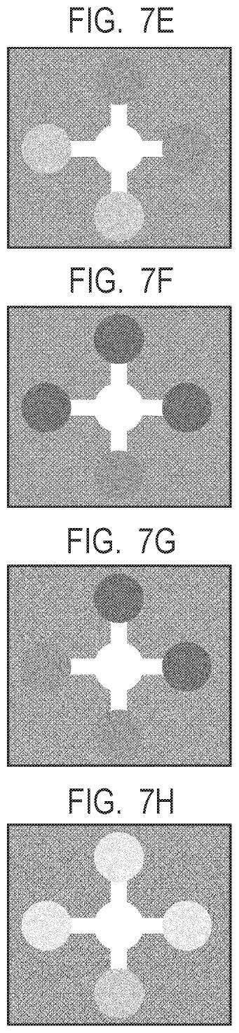

In the case of using the above-mentioned fluidic device, the image patterns of fluorescence intensities or fluorescence changes observed from the detection units of the fluidic device at the time of the addition of the respective thiol compounds are as shown in FIG. 7E, FIG. 7F, FIG. 7G, and FIG. 7H based on the results of Table 2. The case of adding cysteine is as shown in FIG. 7E, the case of homocysteine is as shown in FIG. 7F, the case of glutathione (reduced form) is as shown in FIG. 7G, and the case of methionine is as shown in FIG. 7H.

Methionine and the thiol compounds contained in an unknown specimen can be identified and quantified by comparing a signal pattern emitted from the detection units through addition of the unknown specimen in similarities to the image patterns of FIG. 7E, FIG. 7F, FIG. 7G, and FIG. 7H, and calculating fluorescence intensity values and fluorescence intensity ratios between the detection units, followed by comparison to Table 2.

As described above, it was found that, when the fluidic device according to Examples of the present invention was used, methionine and the thiol compounds were able to be determined without performing component separation.

Simple analysis of a measurement object having a certain functional group has been enabled.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-179857, filed Nov. 9, 2022, and Japanese Patent Application No. 2023-142316, filed Sep. 1, 2023, which are hereby incorporated by reference herein in their entirety.

What is claimed is:

1. An analysis apparatus for analyzing a specimen that may contain one or more kinds of measurement objects,
the measurement objects each being a substance that satisfies at least any one of the following conditions α) and β):
α) having a certain functional group; and
β) being capable of reacting with an enzyme to produce a compound having the certain functional group,
the analysis apparatus comprising:
an addition unit to which the specimen is added; and
a plurality of flow paths communicating downstream of the addition unit, and a plurality of detection units respectively communicating to the plurality of flow paths,
wherein each of the plurality of detection units includes one kind of detection reagent that reacts with the certain functional group,
wherein the detection reagent contains a compound having specific reactivity with the certain functional group, and
wherein the plurality of detection units include two or more kinds of detection reagents different from each other in the reactivity.

2. The analysis apparatus according to claim 1, further comprising a reduction unit communicating downstream of the addition unit and communicating upstream of the plurality of flow paths.

3. The analysis apparatus according to claim 2, wherein the reduction unit is formed of a gel carrier having immobilized thereon tris(2-carboxyethyl)phosphine.

4. The analysis apparatus according to claim 1,
wherein the measurement objects are each a substance that satisfies at least the condition β), and
wherein the plurality of detection units include:
a detection unit including the enzyme; and
a detection unit free of the enzyme.

5. The analysis apparatus according to claim 4, wherein the enzyme is methionine γ-lyase.

6. The analysis apparatus according to claim 1, further comprising:
an information acquisition unit configured to acquire signal information that is information about a signal emitted from each of the plurality of detection units; and
an information processing unit configured to generate information on the measurement objects from the signal information.

7. The analysis apparatus according to claim 6, wherein the information processing unit is configured to generate information on the measurement objects in the specimen from the signal information through use of a reference database.

8. The analysis apparatus according to claim 6, wherein the information on the measurement objects, which is generated by the information processing unit, includes at least any one of information about a type of at least one kind of the measurement objects and information about a presence amount of at least one kind of the measurement objects.

9. The analysis apparatus according to claim 6, wherein the signal information includes at least any one of the presence or absence of the signal of each of the plurality of detection units and an intensity of the signal of each of the plurality of detection units.

10. The analysis apparatus according to claim 6, wherein the signal information includes one or more of a hue, a lightness, a fluorescence intensity, a fluorescence wavelength, a luminescence intensity, and a luminescence wavelength of each of the plurality of detection units.

11. The analysis apparatus according to claim 1, wherein the detection reagent that reacts with the certain functional group reacts with the measurement objects to show one of color development, color change, fluorescence, or luminescence.

12. The analysis apparatus according to claim 1, wherein the certain functional group is a thiol group.

13. The analysis apparatus according to claim 12, wherein at least any one of the detection reagents that each react with the certain functional group has a monovalent group represented by the following formula (1): 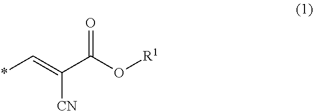
in the formula (1), R¹ represents a hydrogen atom or an alkyl group having 1 or more and 3 or less carbon atoms, and * represents a bonding site.

14. The analysis apparatus according to claim 12, wherein at least any one of the detection reagents that each react with the certain functional group has a monovalent group represented by the following formula (2): 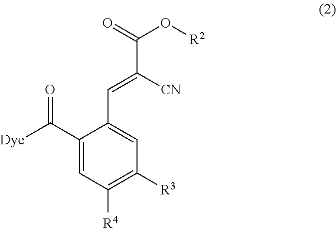
in the formula (2), R² represents a hydrogen atom or an alkyl group having 1 or more and 3 or less carbon atoms, R³ and R⁴ each independently represent a hydrogen atom or a methoxy group, and Dye represents a fluorescent dye having a light absorption band in a wavelength region of from 350 nm to 550 nm.

15. An analysis kit for analyzing a specimen that may contain one or more kinds of measurement objects,
the measurement objects each being a substance that satisfies at least any one of the following conditions α) and β):
α) having a certain functional group; and
β) being capable of reacting with an enzyme to produce a compound having the certain functional group,
the analysis kit comprising a fluidic device including:
an addition unit to which the specimen is added; and
a plurality of flow paths communicating downstream of the addition unit, and a plurality of detection units respectively communicating to the plurality of flow paths,
wherein each of the plurality of detection units includes one kind of detection reagent that reacts with the certain functional group,
wherein the detection reagent is a compound that has chemoselectivity to the certain functional group and reacts with molecules having similar structures to the measurement objects in various reaction degrees, and
wherein the plurality of detection units include two or more kinds of detection reagents different from each other in the reaction degrees.

16. The analysis kit according to claim 15,
wherein the measurement object is a substance that satisfies at least the condition β), and
wherein the plurality of detection units include:
a detection unit including the enzyme; and
a detection unit free of the enzyme.

17. An analysis method for analyzing a specimen that may contain a measurement object,
the measurement object being a substance that satisfies at least any one of the following conditions α) and β):
α) having a certain functional group; and
β) being capable of reacting with an enzyme to produce a compound having the certain functional group,
the analysis method comprising:
an introduction step of introducing the specimen into a plurality of detection units via a plurality of flow paths; and
a reaction step of allowing the specimen to react with one kind of detection reagent that reacts with the certain functional group in each of the plurality of detection units,
the plurality of detection units including two or more kinds of detection reagents that each react with the certain functional group.

18. The analysis method according to claim 17, further comprising, after the reaction step, a step of determining at least any one of a type of at least one kind of the measurement object and a presence amount of at least one kind of the measurement object by visually observing a signal emitted from each of the plurality of detection units.

19. An analysis method for analyzing a specimen that may contain a measurement object,
the measurement object being a substance that satisfies at least any one of the following conditions α) and β):
α) having a certain functional group; and
β) being capable of reacting with an enzyme to produce a compound having the certain functional group,
the analysis method comprising:
a reduction step of reducing the specimen by passage through a reduction unit via a flow path;
an introduction step of introducing the specimen from the reduction unit into a plurality of detection units via a plurality of flow paths; and
a reaction step of allowing the specimen to react with one kind of detection reagent that reacts with the certain functional group in each of the plurality of detection units,
the plurality of detection units including two or more kinds of detection reagents that each react with the certain functional group.

20. The analysis method according to claim 17,
wherein the measurement object is a substance that satisfies at least the condition β), and
wherein the reaction step comprises a reaction step of allowing the specimen to react with the enzyme.
